(19) United States
(12) Patent Application Publication
(10) Pub. No.: US 2016/0206391 A1
(43) Pub. Date: Jul. 21, 2016

(54) A SYSTEM TO MANIPULATE ORGANS AND INSTRUMENTS FOR MINIMALLY INVASIVE SURGERY

(71) Applicant: Chinmay DEODHAR, Kothrud, Pune (CN)

(72) Inventor: CHINMAY DEODHAR, PUNE (IN)

(21) Appl. No.: 15/023,377

(22) PCT Filed: Sep. 23, 2014
(86) PCT No.: PCT/IB2014/064772

§ 371 Date: Mar. 19, 2016

(30) Foreign Application Priority Data
Sep. 24, 2013 (IN) 3059/MUM/2013

(51) Int. Cl. A61B 34/00; A61B 50/30; A61B 17/04; A61B 17/34; A61B 17/29; A61B 17/12

(52) U.S. Cl. CPC A61B 34/74; A61B 17/29; A61B 17/12013; A61B 17/0469; A61B 17/3478; A61B 50/3001; A61B 2017/00991

(57) ABSTRACT

The present invention is generally related to the art of minimally invasive surgery, and more specifically to reduced or single port laparoscopic surgery. This invention is a system of instruments that generally comprises of a surgical end effector, a receiver unit connected to the base of such an end effector, an external control link and an applicator for the control link. The entire system enables a surgeon at the time of his or her choosing, to externally control the end effector to manipulate organs, with minimal interference in the procedure at hand. In a preferred embodiment, the surgical end effector may be a mechanical atraumatic tissue grasping component enabling the retraction of internal organs.

### FIELD OF THE INVENTION

The present invention is generally related to the art of surgical instruments for use in laparoscopic surgery and more specifically to a system of instruments capable of being used in less invasive laparoscopic surgery, such as reduced port laparoscopic surgery, or single incision laparoscopic surgery.

### BACKGROUND OF THE INVENTION

Traditionally, open surgery has been carried out by making a large incision (typically being anywhere between 30 to 300 mm in length) in the abdomen to open up the skin and reach the internal organs where the procedure is to be carried out. With better instrument development, the open surgical methods slowly started getting replaced by minimally invasive surgical methods, ie laparoscopic surgery. In laparoscopic surgery, instead of one large incision, several small incisions are made, typically 5 mm, 8 mm, or 10 mm in diameter. Long thing tools are inserted through special ports that are placed in these holes. This surgery limits the invasiveness to just 4-5 incisions in the abdominal fascia, thus also reducing the postoperative pain, rates of complication, amount of bleeding and many other benefits, already known in the art.

Of late, there have been discussions on further reducing the number and size of incisions to perform surgery. Studies have indicated that reducing the size of incision and/or the number of incisions may lead to better cosmetic results for patients, lower post-operative pain, faster recovery, reduced chance of port site complications and reduced operative time.

Some simpler procedures such as appendectomy, cholecystectomy, etc. have already been performed with fewer incisions, or in some cases just a single incision at the navel, thus further improving cosmesis and providing other benefits. This is because, in place of 4 to 5 incisions, only a single incision is sufficient.

However, currently only some simple procedures can be performed by the single incision method since techniques and instruments to perform more sophisticated procedures are still in early stages of development. Some of such evolving ideas are disclosed in the prior art.

US2001282159 discloses a technique wherein a retracter clamp is inserted into the abdomen using one of the ports at the navel. The retracter clamp is connected to a hook. The clamp holds on to the organ that has to be retracted, following which the hook is attached to the internal surface of the abdominal wall, thus pulling the clamped organ upward. This technique achieves organ retraction with no external scars. Other similar retraction techniques exist which retract organs completely internally. Due to the use of clamps, the organ retraction is atraumatic.

However, one limitation of these techniques is that once the organ is retracted, it is cumbersome to change the position or degree of retraction of the organ. This is because there is no external control over the position of the clamp. The surgical instruments themselves are used to maneuver them, for which the procedure at hand must be temporarily stopped. Further, in order to reposition the endo-clamp, a special type of grasping device has to be utilized, as ordinary instruments cannot be used.

Another limitation of these techniques is also that they require the internal clamp to grip the retracted organ with a force exerted by an internal spring. This is because in the other techniques, no external method exists to control the amount of force necessary to grip the organs.

Some other retraction methods include suture-needle retraction, wherein a particular organ that is to be removed, such as the appendix in an appendectomy procedure or the gall bladder in the cholecystectomy procedure can be retracted by driving a needle from outside, through the respective organ and again out of the abdomen. This method of retraction is traumatic to the organ that is to be retracted, and in some cases can cause problems such as leakage of bile (in case of cholecystectomy). Organs that are not to be removed, but need to be retracted such as intestines, liver, etc. cannot be retracted using this method as they would get damaged.

### OBJECTS OF THE INVENTION

It is an object of this invention to provide for a method and a system of instruments that allow organ retraction in minimally invasive laparoscopic surgery, such that the retracted organ can be externally controlled and the clamping of the organ is atraumatic.

It is another object of this invention to minimize the thickness of the incision necessary to achieve such external control (eg: up to 1 mm in diameter or less).

It is yet another object of this invention to provide multiple directions from which a clamp could be controlled, in order to change or adjust the direction of retraction during surgery.

It is yet another object of this invention to provide for a system that allows the surgeon to decide on-the-go where and how many directions from which to externally control an internal clamp to retract organs.

It is another object of this invention to provide for a system where the internal clamp does not require a specialized grasper to re-position it, but ordinary instruments already inserted for carrying out the procedure at hand can do this job.

It is yet another object of this invention to provide for a method and system of instruments to enable the manipulation of internal organs as well as manipulation of other devices inside the body cavity by control links that extend outside the body cavity.

### SUMMARY OF THE INVENTION

This invention generally comprises of a surgical end effector, a receiver unit connected to the base of such an end effector, an external control link and an applicator for the control link.

In a preferred embodiment of the invention, the surgical end effector may be a mechanical atraumatic tissue grasping component.

In a preferred embodiment of the invention, the surgical end effector along with the attached receiver unit, are initially on a first side of a tissue surface while the external control link and applicator are initially on a second side of the tissue surface, where the second side may be opposite to the first side of the tissue surface.

In another embodiment of the invention, there may be multiple surgical end effectors, each with at least one receiver unit connected to their base, and several external control links attached by means of one or more control link applicators.

In a preferred embodiment of the invention, the mechanical atraumatic tissue grasping component may be an endo-clamp capable of grasping tissue. In other embodiments, it may be a clamp with serrations, or a grasper similar to a bull-dog clip. This clamp may be designed to grasp the tissue or internal organs that need to be retracted in an atraumatic manner. Graspers come in a wide variety of functions and types for various purposes. These include tissue, claw, sharp tooth, endo clinch, alligator, aggressive, cobra tooth, spoon, cup, bab cock, DeBakery, Allis graspers, bulldog clamp and so on.

In a preferred embodiment of the invention, the external control link may be a suture of any suitable material and thickness, such that it is strong enough to sustain the forces of retraction. In another embodiment it may be a thread, or any other thin filament-like structure capable of sustaining retraction forces.

In a preferred embodiment of the invention, the control link applicator may be a cannula, with a sharp tip and a very thin diameter. In other embodiments, the control link applicator may be a cannula with a specially designed sharp tip at front on which the control link may be positioned. Some of these embodiments are shown in the figures. In yet another embodiment, the control link applicator may be a needle. The needle may have several alternative design embodiments and features that allow the control link to be positioned on the body of the applicator. Some of these features, in some embodiments may enable the control link to be smoothly transferred to the receiver unit when the applicator is inserted into it.

In a preferred embodiment of this device, the receiver unit connected to the clamp is designed so as to accept the control link applicator and smoothly transfer the external control link from being attached to the applicator, to being secured onto the receiver unit, such that when the applicator is withdrawn, the external control link can exert a force on the receiver unit, which in turn further exerts a force on the surgical end effector, such as an endo-clamp.

In yet another embodiment of this invention, the external control link and control link applicator may be contained in an applicator housing, which will make it easy for the user to insert and eventually withdraw the applicator in a safe and convenient manner. Such applicator housing may also further contain a mechanical means for protruding and retracting the external control link. It may also contain mechanical means for protruding and retracting the control link applicator. This applicator housing may be ergonomically shaped so as to enable the user to handle the enclosed elements in a convenient fashion. In some embodiments, this housing may take the shape of a writing pen and may include gripping features. In yet another embodiment, such an applicator housing may contain motorized components for motion of the applicator as well as the control link, such that these motions may be controlled electronically from a console.

This entire apparatus is useful in reducing the number of ports required for general laparoscopic surgeries. It may be especially useful where reduced port surgeries are currently not possible due to lack of instrumentation for atraumatic retraction, capable of being controlled externally, on-the-go. For example, once the endo-clamps are inserted into the abdominal cavity, the surgeon may decide at his or her discretion, when to commence retraction. At the time of his or her choosing, the endo-clamp may be connected to one or more external links and then attached to an internal organ very easily, without losing the train of thought of the main surgery at hand.

Further, other types of surgical end effectors may also be similarly used by the surgeons and manipulated externally like the example of the endo-clamp for organ retraction described above.

### BRIEF DESCRIPTION OF THE DRAWINGS AND ELEMENTS

The invention will now be described here below with the help of drawings and diagrams provided with this application.

FIG. 1 shows the overall view of the entire system, as it is envisaged to be utilized in one of the embodiments.

FIG. 2 shows one embodiment of an applicator for external control link which takes the form of a needle, containing special features at the tip. These features include an eye for a loop of the external control link.

FIG. 3 shows yet another embodiment of an applicator with an external control link attached to it in a particular fashion. This applicator is of the needle form factor.

FIG. 4 shows yet another embodiment of an applicator with an external control link attached to it. This applicator is also of the needle form factor.

FIG. 5, FIG. 6, FIG. 7, FIG. 8 and FIG. 9 show further different embodiments of the applicator along with various elements to attach external control links to them. All of these are of the needle form factor.

FIG. 10, 11, 12, 13, 14, 15, 16, 17, 18, 19, 20 show different embodiments for the applicator along with various elements on which the external control links get attached. These are all of the form of a cannula.

FIG. 21 shows one embodiment of a receiver unit which is being actuated by one of the applicator embodiments, in order to smoothly transfer the external control link from the applicator to the receiver unit.

FIG. 22 shows this embodiment of the receiver unit (same as that shown in FIG. 21) after the transfer of the external control link has been completed. It further illustrates one embodiment of the tissue grasping component, such as an endo-clamp.

FIG. 23 shows one embodiment of a part of the receiver unit which allows a smooth transfer and locking-on of the external control link from the applicator to the receiver unit.

FIG. 24 shows further components of the assembly of the receiver unit along with features that enable the smooth transfer, as in FIG. 23.

FIGS. 25, 26 and 27 show further different embodiments of various designs of receiver units. Each of these illustrate different mechanisms with which an applicator such as a needle, cannula or others illustrated earlier may conveniently transfer the external input links onto the receiver units.

FIG. 28 shows another embodiment of a receiver unit, where it is of the form of a semi-rigid rubber-like material through which a sharp pointed applicator may be inserted. This material may be a soft silicone, bio-compatible polymer, natural polymer or any similar material with a characteristic clay-like softness.

FIGS. 29 and 30 illustrate the rubber-like material where various designs of applicators have attached the external control links into it.

FIG. 31 illustrates yet another different embodiment of a receiver unit.

FIG. 32 illustrates a mechanism which can be used to transfer the external input link from the applicator to a receiver mechanism. A different form of this mechanism is used in other embodiments of the receiver units.

FIG. 33 illustrates another embodiment of the receiver unit where the external control link gets automatically transferred from the applicator to the receiver unit with the help of a mechanism with a characteristic cam-like surface, slots, a spring and a stopper.

FIG. 34-A illustrates the mechanism described in FIG. 33, but with a slot and cam surface of a different design and shape. It may be noted that instead of such a cam mechanism at the receiver unit, a similar, but an inverse design of the same mechanism may be used at the other end of the applicator.

FIG. 34-B, FIG. 34-C and FIG. 34-D further show different embodiments of parts shown in FIG. 33 and FIG. 34-A.

FIGS. 35, 36 and 37 show the step wise working illustrations of the mechanism by which the external control link gets smoothly and conveniently transferred from the applicator to the receiver unit.

FIG. 38 illustrates one embodiment of a mechanical atraumatic tissue grasping component. This is similar to a bulldog clamp.

FIGS. 39 and 40 illustrate another embodiment of the mechanical grasping component, in the jaw open and closed positions respectively. These figures further illustrate one mechanism with which the jaws of this particular design are closed and opened. This is similar to the mechanism of a clicking ball-pen with a push button.

FIG. 41 illustrates a grasper-like instrument being used to open or close the jaws of a bulldog clamp-like grasping component.

FIGS. 42, 43, 44 and 45 illustrate other methods by which the jaws of the second type of the grasping component may be opened and closed.

FIG. 46 illustrates one embodiment of a pen-like applicator housing that contains the applicator, the external control link, and various other features and elements to enable the user to handle these components easily.

FIGS. 47 and 48 illustrate yet another embodiment of the tissue grasping mechanism where a ratchet-like mechanism can be used tighten and lock the grasper onto the tissue when pulled by the external control link attached to it via the receiver unit. Here, the jaws of the grasper may be of a flexible compliant material such as polypropelene, or another flexible plastic.

FIGS. 49 and 50 illustrate the how the ratchet mechanism may be unlocked with help of another grasper inserted through a regular port.

FIGS. 51 and 52 illustrate yet other embodiments for the tissue grasping mechanisms which may be locked by a ratched-like mechanism.

FIG. 53 illustrates the use of the apparatus where one or more receiver units are attached to a flexible surface or a band, such as a silicone band and can be used to retract organs.

FIG. 54 illustrates the use of the apparatus where one or more receiver units are attached to a rigid rod such that the assembly could be used to retract large or heavy organs. Further, the rigid rod may be telescoping in nature, such that a wider range of size of organs may be retracted.

FIG. 55 illustrates the use of the apparatus where the receiver unit is used along with an endoscope with a channel for one or more instruments. This may be in the gastrointestinal tract, blood vessels, trachea, or other hollow cavities within the body which may need surgical intervention.

FIGS. 56 and 57 illustrate the use of the apparatus along with various other types of instruments, such as a scissor, grasper, clip applier, specimen retrieval bag, or other such devices which may be controlled via the external control link in combination with other instruments.

FIG. 58 illustrates the use of the apparatus to hoist and control a camera component in reduced or single port laparoscopic surgery.

FIG. 59 illustrates the use of the apparatus to hoist and control a fluid flow instrument, such as a suction or irrigation tube.

FIG. 60 represents various components which enable the surgeon to use this apparatus in a safe and convenient manner, while using only one hand, such that the other hand may be used to manipulate the camera or other instruments.

The invention may be more fully understood by reference to the cited figures and details of exemplary embodiments. Alternative embodiments of the invention as claimed, and providing the benefits of the novel concepts of the invention, are contemplated and will be obvious from the explanations hereinafter.

### DETAILED DESCRIPTION

Minimally invasive techniques, and in particular, laparoscopic surgical methods have offered many benefits to patients. As the methods and instruments improve, techniques get continuously developed to achieve more and more procedures in ever fewer number of incisions in the body. Some procedures, such as appendectomy, cholecystectomy and others can even be performed via a single incision at the belly-button.

However, in single incision or reduced port surgery, a major challenge that remains even today, and which is still not adequately addressed by devices in the prior art, is that of organ retraction. The inventive step in the present technology involves the method and mechanism by which a quick and easy connection is formed between a surgical end effector and an external control link. In a preferred embodiment, the surgical end effector may be an endo-clamp, while the control link may be a suture length, such that an organ which the endo-clamp grips can be retracted by pulling on the suture that is outside the abdomen. The suture can be held in position by clips.

We begin the detailed description of this invention by referring to FIG. 1, where an overall view of the entire system is represented, generally indicated by reference number 001. It shows a tissue surface, generally indicated by reference number 013. In one embodiment, this surface may be the abdominal fascia, where FIG. 1 indicates the side sectional view of an inflated abdomen of a patient during a laparoscopic surgery. The operative area, generally indicated by reference number 014, includes several organs. Also shown are some surgical instruments generally indicated by reference numbers 010, 011 and 012. In a typical reduced port laparoscopic surgery, various instruments are inserted through one or more ports, generally indicated here by reference number 009.

Also shown in FIG. 1 is a mechanical atraumatic grasping component, such as an endo-clamp, generally indicated by reference numeral 002. Several different embodiments of such an endo-clamp exist, such as tissue, claw, sharp tooth, endo clinch, alligator, aggressive, cobra tooth, spoon, cup, bab cock, DeBakery, Allis graspers, bulldog clamps, and so on. A receiver unit is generally indicated by reference number 003. The organ which is gripped by the endo-clamp, and which is shown in a partially retracted position, is generally indicated by reference number 004. A first external control link, such as a suture, is generally indicated by reference number 005. It is shown attached to the receiver unit 003. A second external control link, which may also be a suture is generally indicated by reference number 006. It is shown attached to the same receiver unit 003. However, in other embodiments, external control links may be attached to other receiver units, attached to other endo-clamps, during the same procedure. It may be noted that both control links extend across the tissue surface 013, thus being able to transmit a force from one side of the tissue surface to the other. In a preferred embodiment, this tissue surface may be the abdominal fascia, as represented by FIG. 1, showing a typical laparoscopic surgery, where the control links may extend outwards through the skin and outside the body of the patient, whereby a force exerted on these links outside the body is transmitted inside the body, via the link, receiver unit and the grasping component, ultimately being able to retract the organ 004. Clips, which are generally indicated by reference numbers 007 and 008 are attached to the sutures outside the body, to hold them in place, where they keep the gripped organ retracted.

A typical procedure using this system may proceed as follows: as the surgery begins, endo-clamp 002 is introduced from one of the ports at the incision 009, along with the instruments. Next, at a time and position of the surgeon's choosing, an applicator (which will be described subsequently) is introduced from the external surface of the skin, from outward to inward direction. The applicator engages with the receiver unit of the endo-clamp and conveniently attaches to it a control link such as a suture, 005. As the applicator is withdrawn from the body, it leaves behind the suture attached to the endo-clamp's receiver unit, extending all the way out of the body of the patient. Next, using the existing instruments 010 or 012, the endo-clamp is attached to organ 004 which is to be retracted.

In this position, the surgeon can retract the organ by pulling the suture outward. Once the desired extent of retraction is achieved, the surgeon may attach a clip 007 to the suture just outside the skin surface, to keep the suture locked in the position, and hence the organ retracted.

If, during the procedure, the surgeon wishes to adjust the position of the endo-clamp, in another direction, he or she may introduce another applicator from a different angle and attach another suture 006 to the same endo-clamp's receiver unit. Pulling on this second suture in its direction will result in the endo-clamp being adjusted in the desired direction. This second suture may be fixed to the body by means of another clip 008. Similarly, a third or more sutures may also be added as needed onto the same receiver unit.

It may be noted that in this entire procedure, extremely thin punctures are sufficient, just enough to let the applicator and suture pass. This may typically be less than 0.75 mm, although other dimensions are also possible. Such tiny incisions leave a very negligible scar, as compared to other methods which use 2 mm to 5 mm diameter incisions. Further, the size of the surgical end effector such as an endo-clamp, may have the full 5-10 mm width to allow atraumatic retraction. At the same time, this method is extremely easy and convenient to use, due to the mechanism employed to attach the external control link to the endo-clamp's receiver unit, by the applicator. This forms the core inventive step of this system.

Various embodiments for the applicator, the external control links, the receiver units as well as the endo-clamp are disclosed subsequently.

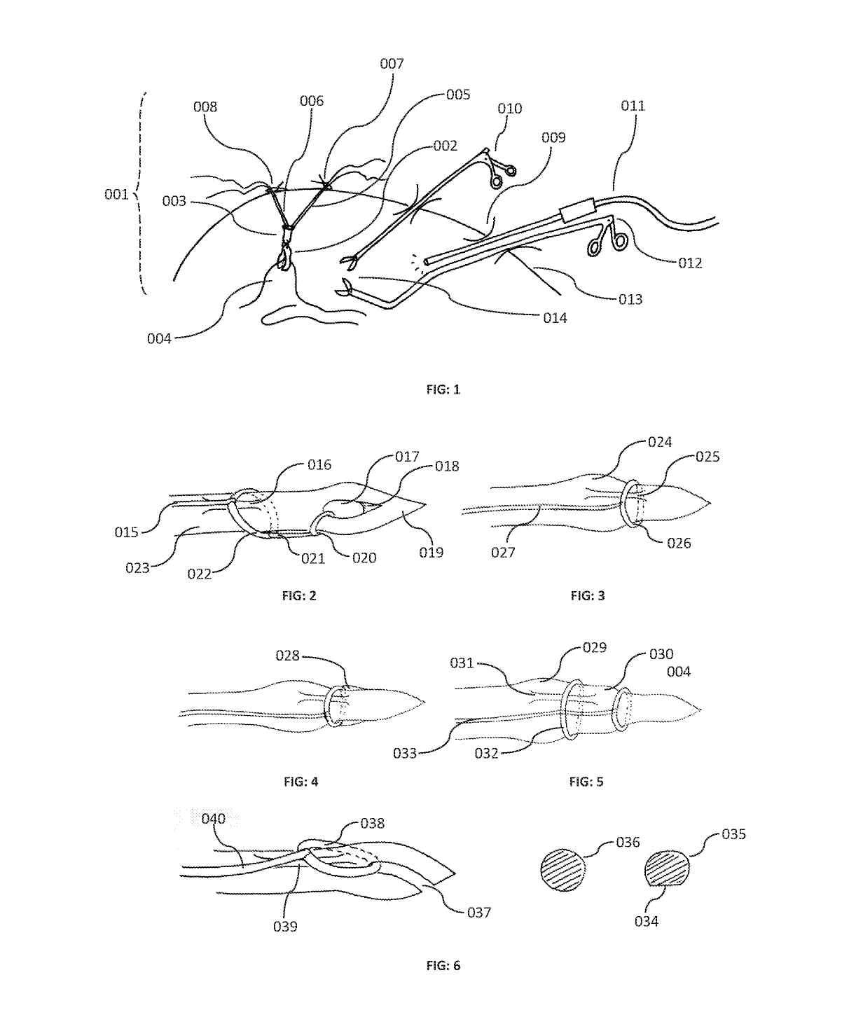

Shown in FIG. 2 is one embodiment of the applicator that takes the form factor of a needle, indicated by reference number 023. This needle has characteristic features, such as an eye, generally indicated by reference number 017, a groove indicated by reference number 016, a cutting blade generally indicated by reference number 018 and a sharp front tip generally indicated by reference number 019. An external control link in the form of a suture is generally indicated by reference number 015. It is attached to the needle by two loops, generally indicated by reference numbers 020 and 022. These loops are connected to each other by a length of suture indicated by numeral 021.

Shown in FIG. 3 is another embodiment of the applicator. This is characterized by features such as a groove indicated by reference number 025 and a thicker portion generally indicated by reference numeral 024. The external control link is in the form of a suture, indicated by reference number 027, which forms a loop 026, and is attached to the needle beyond the thicker portion 024. The thick portion prevents the loop from sliding backwards as the needle is inserted through the skin.

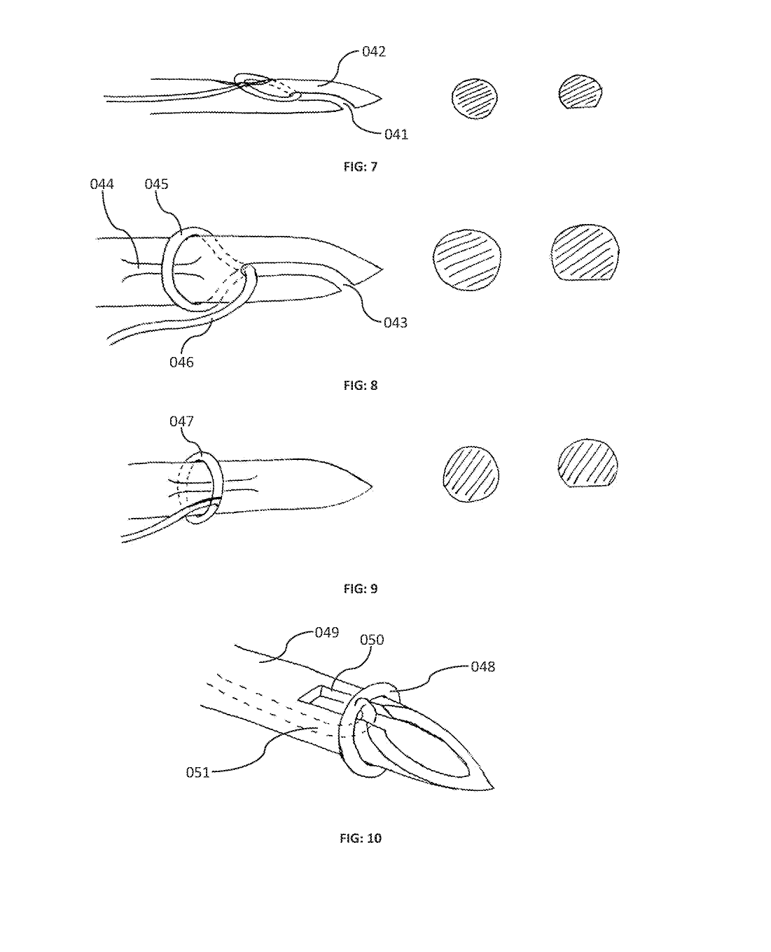

Shown in FIG. 4 is another embodiment of the applicator in the form of a needle, also with a thicker portion, a groove and fins. The fins are generally indicated by reference number 028. The fins help guide the applicator across the skin smoothly

Shown in FIG. 5 is yet another embodiment of the applicator, again in the form of a needle, but this time with two different thick portions of increasing diameters, generally represented by reference numbers 030 and 029. A groove 031 is also illustrated, over which a loop 032 is formed of the suture 033.

FIG. 6 shows a different embodiment of the applicator, having a slit cut near its tip in a diagonal fashion. This slit is generally represented by reference number 037. An external control link in the form of a suture is generally represented by reference number 040. This suture has a loop at the end, which is represented by reference number 038. The loop 038 is wound across the head of the needle through the slit 037. This needle also has a groove below, generally represented by reference number 039. Shown on the side are different possibilities of the cross section of the needle. It may be circular, as represented by reference number 036, or it may have a flat bottom indicated by number 034, and rest of it to be rounded, indicated by reference number 035. The flat side 034 helps to align the needle correctly into the receiver unit.

FIG. 7 illustrates another embodiment of the applicator in the needle form. This variation has very similar features to the needle illustrated in FIG. 6, except that in the present example of FIG. 7, the tip does not have a higher thickness or a bulge, but instead is a uniform smooth cross section. Again, the overall cross section may be circular or ‘D’ shaped, with one side flat, to enable easy alignment.

FIG. 8 shows yet another embodiment of the applicator, in the form of a needle. The key features on the needle are similar to those found in FIG. 7. However, the external control link in the form of a suture indicated by reference number 046, attaches to the needle via a loop indicated by reference number 045. The loop is wound over a groove, indicated by reference number 044. Again, the cross section of the needle may be circular or it may be a ‘D’ shape.

FIG. 9 shows another embodiment of the applicator in the form of a plain needle, with only a groove as its feature. The external control link, in the form of a suture is wound around its diameter in the form of a hardened loop, generally indicated by reference number 047. The loop stays in place by means of friction. The cross section may be circular or it may be a ‘D’ shape.

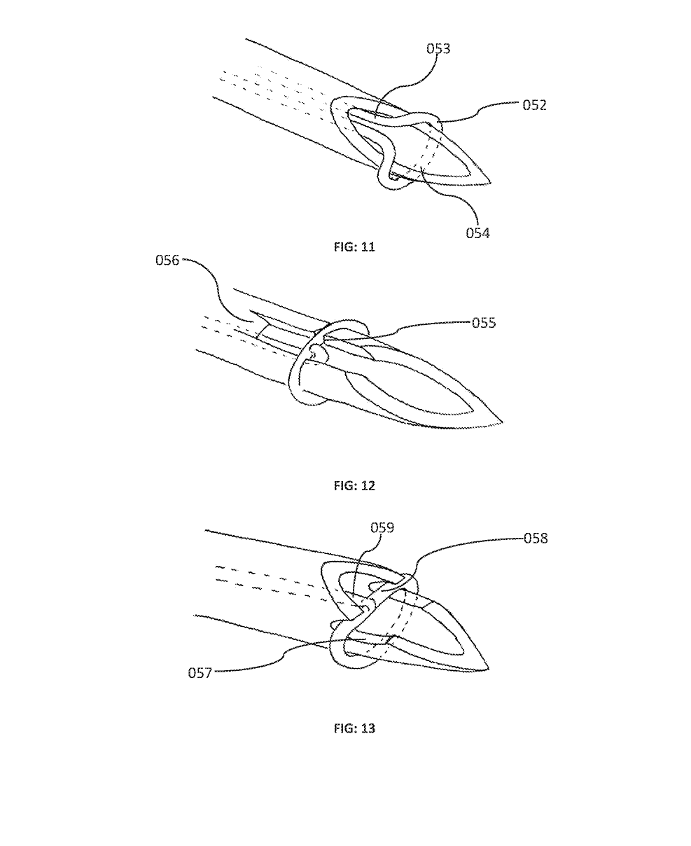

FIG. 10 illustrates another embodiment of the applicator, in the form of a cannula, generally indicated by reference number 049. The cannula has a slit cut longitudinally, generally indicated by reference number 050. The external control link, in the form of a suture is generally indicated by reference number 051. It passes along the internal cavity of the cannula, rising up through the slit 050, and forming a loop 048 around the diameter of the cannula.

Yet another embodiment of the applicator in the form of a cannula is represented in FIG. 11. The external control link in the form of a suture is routed in a specific manner, such that it runs along the length of the cannula, inside it, as indicated by reference number 053. Towards the tip, it circles around the edge, generally indicated by reference number 052. It moves along the back side of the cannula, depicted by reference number 054 and is wound around again to flow back into the internal cavity of the cannula. As will be described subsequently, the loop 054 would be utilized by various receiver unit mechanisms to hook the suture onto the receiver units.

Another embodiment of the applicator is illustrated in FIG. 12, in the form of a cannula again. This is very similar to the cannula shown in FIG. 10. Some differences include the smooth slope as illustrated at the base of the slit and indicated by reference number 056. Another difference includes the groove along the circumference of the cannula, which is represented by reference number 055, and into which the loop of the suture is secured.

FIG. 13 illustrates another applicator embodiment, in the form of a cannula, but with a different set of slits. In this embodiment, there are two slits, which may be symmetrical, one of which is generally indicated by reference number 057. The loop of the suture, indicated by reference number 058 is wound around these slits, which help it secure on the tip of the cannula. The length of the suture, indicated by reference number 059 is passed along the internal cavity of the cannula.

FIG. 14 illustrates another embodiment of the applicator, in the form of a cannula. It comprises of a suture length forming the external control link, indicated by reference number 062. At the tip of the cannula, rests a rigid ring, indicated by reference number 061, whose dimension is such that it does not get pulled into the cavity of the cannula. The suture forms another loop indicated by number 060, which is attached to the rigid ring 061.

FIG. 15 illustrates another embodiment of the applicator, in the form of a cannula, with a separable pointed tip. The cannula is represented by reference number 064, while the separable tip is indicated by reference numeral 063. The tip is positioned into the cannula cavity by a protrusion indicated by reference number 065. The protrusion 065 is also connected to the external control link in the form of a suture, indicated by reference number 066, which runs along the internal cavity of the cannula.

Illustrated in FIG. 16 is an embodiment of the applicator where the construction is very similar to that shown in FIG. 11. Further to that, the loop 067 may be hardened to form a rigid curvature, which fits perfectly around the circumference of the cannula, where it is positioned.

FIG. 17 shows another embodiment of the applicator, indicated by number 071, where a tooth-like structure is placed near its tip. This structure, represented by reference number 069 is designed such that it does not fully fit into the internal cavity of the cannula, and hence remains stuck at the tip. The external control link in the form of a suture, indicated by reference number 070 is connected to the tooth-like structure.

FIG. 18 shows yet another embodiment of the applicator, in the form of a cannula with a split tip. The tip, indicated by reference number 075 contains a structure running across its internal cavity, represented by reference number 073. The external control link, in the form of a suture, which is represented by number 074, is attached to the tip 075, for eg, by being wound around the structure 073. The main body of the cannula, represented by reference number 076, has a front portion that is chamfered, as illustrated by reference number 072-a. The back edge of the tip has an abrupt vertical face, indicated by reference number 072-b.

FIG. 19 illustrates another type of an applicator design, in the form of a cannula. The tip of the cannula has two horizontal members generally indicated by reference numbers 078 and 079. These two members split the front opening of the cannula into 3 segments. The external control link, in the form of a suture runs along the length of the cavity of the cannula, coming out of the top segment, moving across the middle segment and finally entering back into the cavity of the cannula running back along its length. This suture is generally indicated by reference number 077.

Illustrated in FIG. 20 is another embodiment of the applicator, in the form of a rigid cannula, generally indicated by reference number 082. The external control link in the form of a suture is indicated by reference number 080. The cannula has a proximal end and a distal end. The suture is bent into two sections at the distal end of the cannula. A first section of the suture runs internal to the cannula along its hollow cavity, while a second section runs along its length on the outer side. The point on the cannula at which the suture is bent into two sections is generally indicated by reference number 081. In some embodiments, this point may be smoothened in order to prevent wear and tear of the suture, as it may rub on it.

In the above paragraphs, we have seen several different embodiments of the applicator, and the ways in which the external control link, usually in the form of a suture, is attached to the tip. Various features at the tip of the applicators help in smoothly transferring the suture to the receiver units. In the subsequent paragraphs, we will see some embodiments of different receiver units and the actual mechanism with which this transfer takes place.

Illustrated in FIG. 21 is one embodiment of a receiver unit. It comprises loop indicated by reference number 087, which turns forward into a pointed tip, indicated by reference number 086. The loop 087 runs around to the other side to a point indicated by reference numeral 084, where it can be attached to a surgical end effector. A hinge, represented by number 085 is situated at the base of the receiver unit. A spring loaded flap indicated by reference number 083 is shown to be pushed open by the forward action of the needle. This causes it to move away from the tip 086. This is the resultant position, when the surgeon has inserted the applicator into the abdominal cavity and has inserted it into the receiver unit, in order to transfer the suture onto it. During the reverse motion of the needle, this tip 086 slides along the groove 016, and gets tucked under the loop 022.

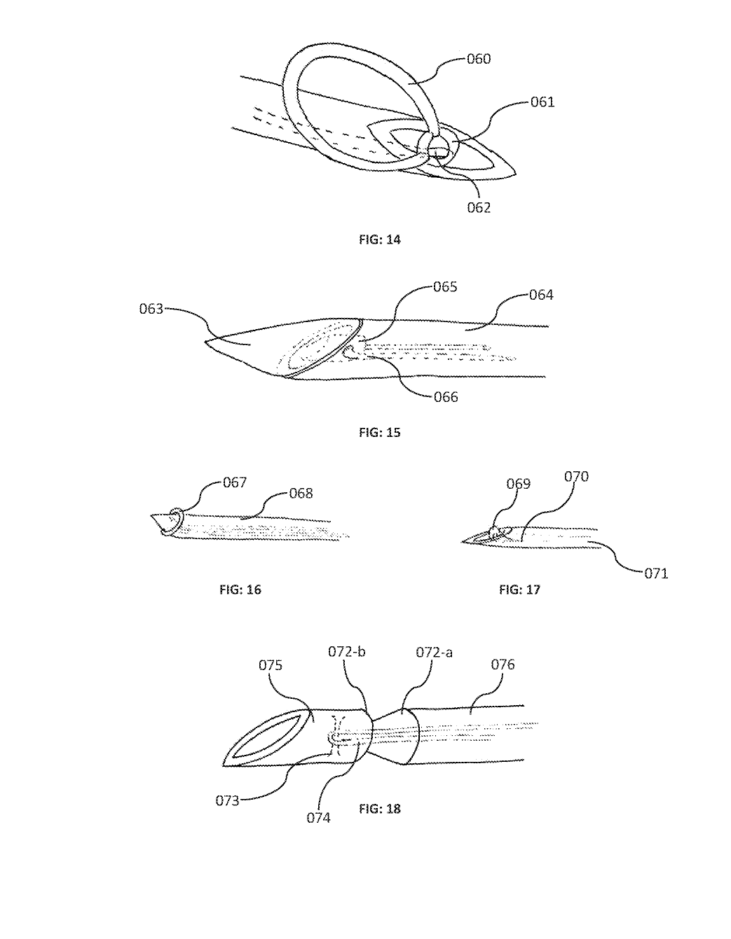

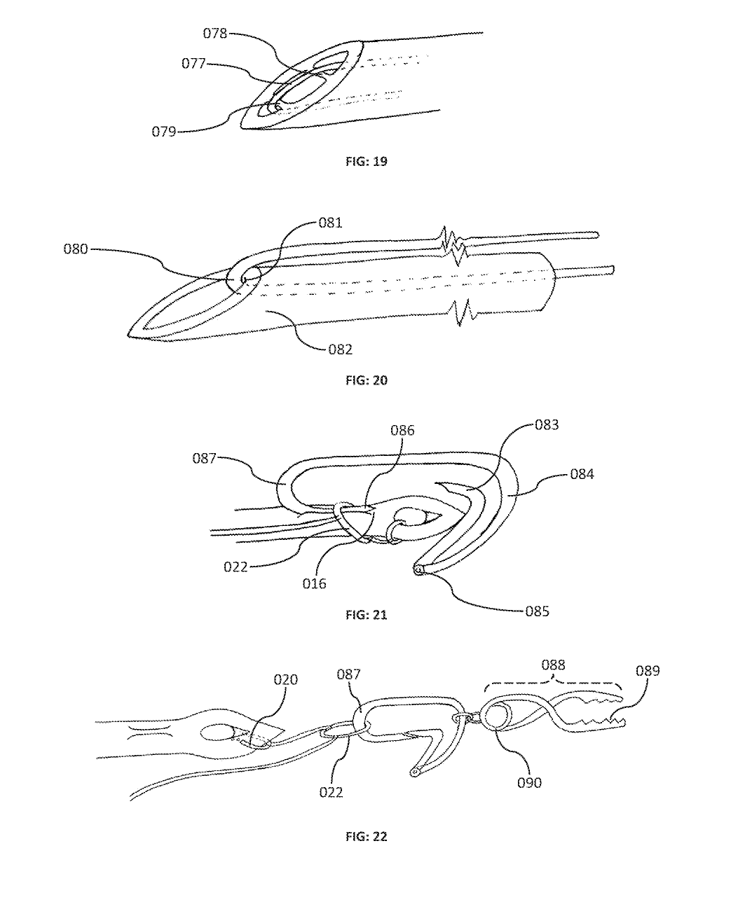

As the needle is pulled back further, the loop 022 smoothly slides along the portion 087, as illustrated in FIG. 22. When the needle is pulled back sufficiently, the loop 020 gets cut off due to the cutting edge 018. Thus, the needle is completely pulled away leaving the suture attached to the receiver unit's segment 087. Also shown in this figure is the endo-clamp indicated by reference number 088. The clamp may have various type of designs, such as serrations, as shown by 089. It may have a spring mechanism at its base in order to grip the tissue or organ. This is illustrated by reference number 090. The endo-clamp grips the organ to be retracted, while the suture gets connected to the other side of the receiver unit, thus completing the entire assembly, which can then be controlled by the surgeon externally by pulling on the other end of the suture.

Thus, we see from the above paragraphs how the end objective of organ retraction can be fulfilled by these embodiments of this invention. Even though the mechanism is described with the help of the applicator embodiment shown in FIG. 2, it must be noted that the same principle may be used on applicators from other described embodiments as well. Some more embodiments of receiver units are described below.

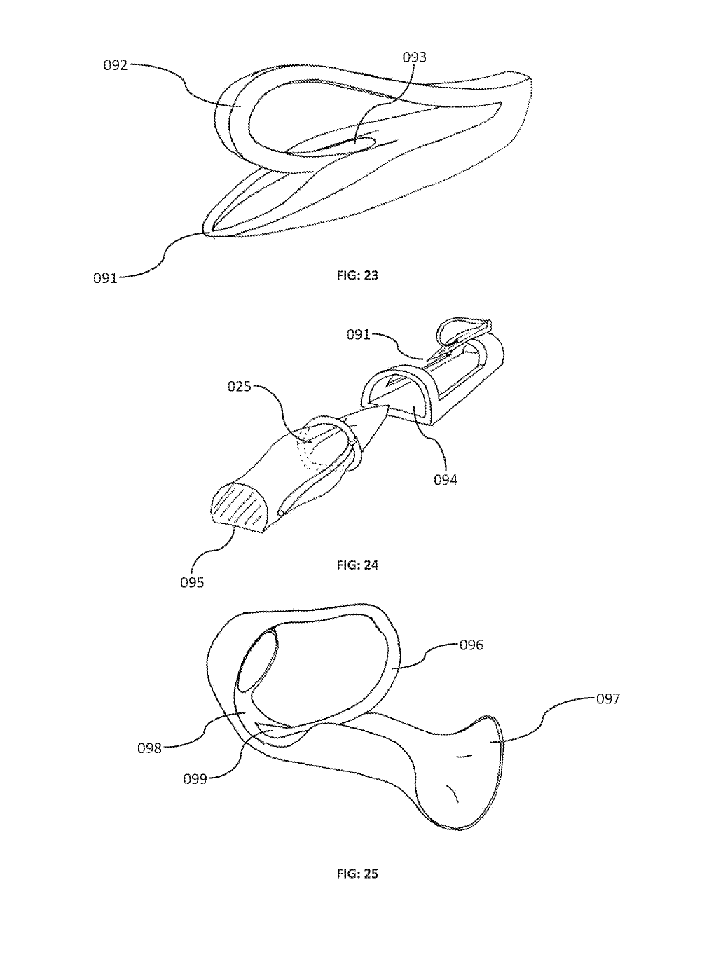

Illustrated in FIG. 23 is another embodiment of the receiver unit, comprising a pointed tip generally illustrated by reference number 091. A ‘J’ shaped hook is bent over it as indicated by reference number 092. The pointed end of the hook is generally indicated by reference number 093.

FIG. 24 sketches a general assembly of the concept illustrated in FIG. 23. The assembly consists of a cavity where one side may be flattened, indicated by reference number 094, to yield an overall cross section of a ‘D’ shape. This makes it possible to align the incoming applicator perfectly along the right direction, since in some embodiments, the cross section of the applicator too is ‘D’ shaped, as illustrated by number 095. As the applicator slides forward, the tip 091 enters the groove 025, and smoothly gets tucked below the loop of the applicator, as shown in the figure. As the applicator is pushed further, the loop snaps past the J shaped hook, beyond the point 093. Once the applicator has reached the furthest point, the surgeon begins to pull it back, thus getting the suture loop attached into the hook, at the portion 092.

As we can see several of the embodiments of the applicator described above may utilized this method of receiver unit.

FIG. 25 shows another embodiment of a receiver unit, which also has a J hook, illustrated by reference number 096. In this embodiment, the base of the receiver unit, indicated by number 098, itself bends upwards to join the tip of the J hook, which is generally indicated by reference number 096. Thus, when an applicator is inserted, the receiver unit bends away to open up the unit, thus allowing the applicator to slide forward till the end. When the surgeon begins pulling the applicator backwards, the tip 099 slides into the respective grooves, getting tucked under the suture loop. As the applicator is pulled back further, the loop gets hooked onto the structure 096. To enable the surgeon to easily pass the applicator into the receiver unit, it has a smoothly splayed opening, generally indicated by reference number 097.

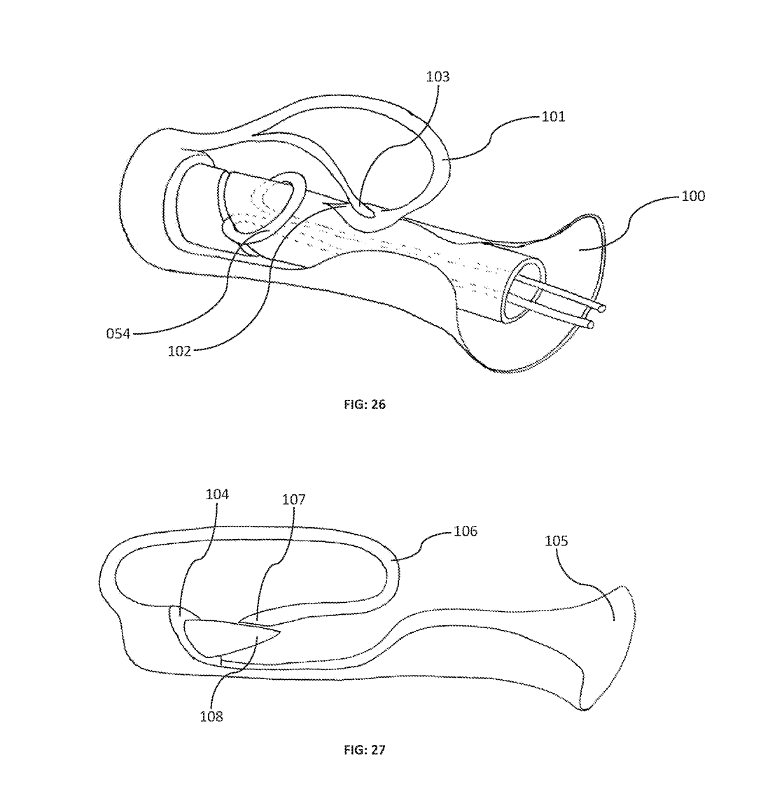

Illustrated in FIG. 26 is another embodiment of a receiver unit, where the elements similarly enable a surgeon to quickly and easily transfer the suture from the applicator to the receiver unit. The position illustrated corresponds to the point where the surgeon has fully inserted the applicator into the receiver unit. As the surgeon pulls the applicator back, the the tip of the J hook of the receiver unit, generally indicated by reference number 102 smoothly slides below the loop 054 of the suture. As the applicator is pulled further back, the loop snaps past the locking element 103 and gets looped around the portion indicated by reference number 101. To enable easy placement of applicator inside the receiver unit by the surgeon, this embodiment may also have a smoothly splayed opening, generally indicated by the number 100.

Shown in FIG. 27 is another embodiment of a receiver unit, which is similar to the one illustrated in FIG. 26. However, in this case, the incoming cannula gets lodged over the protrusion which is generally indicated by reference number 108. At the extreme inward position, it rests against surface 104. In this position, the tip of the hook indicated by number 107 is slightly lifted, to rest upon the outer circumference of the cannula. As the surgeon pulls the applicator backwards, the point 107 smoothly slides through the groove, and under the suture loop. Once the applicator is pulled completely out, the loop gets attached around the part illustrated by reference number 106.

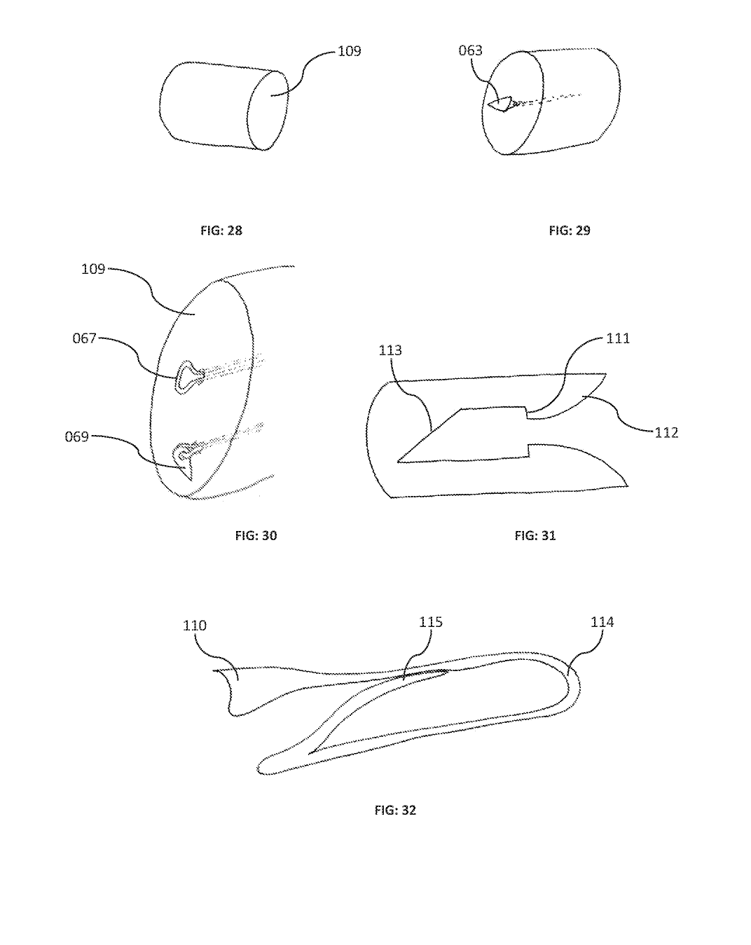

Illustrated in FIG. 28 is a different type of a receiver unit embodiment, which is made of a rubber-like soft material. It may be made out of various other options such as silicone, biocompatible plastic, etc. As shown further in FIG. 29, some embodiments of the applicator are designed to work with this type of a receiver unit. Shown in FIG. 29 is the split type applicator, with its tip 063 stuck on the other side of the unit, when the cannula is pushed through by the surgeon across the unit and then pulled back. Along with the tip, the suture also gets stuck to the piece, thus getting attached to the endo-clamp as well.

Shown in FIG. 30 is the end position of various other embodiments of applicators such as the hardened loop 067, or the tooth-like structure 069 that remain stuck on the other side of the rubber-like material, when the cannula containing these elements is simply inserted and retrieved from the material. Thus, in these cases, we see that the surgeon merely has to push the applicator in the material and retrieve it, which results in the suture getting attached to the endo-clamp.

Illustrated in FIG. 31 is the receiver unit that goes along with the applicator shown in FIG. 18. As the surgeon pushes in the applicator, the flanges generally indicated by reference number 112 pry apart, allowing the cannula to enter. As soon as the tip of the front portion touches the internal surface 113, the receiver unit snaps inward such that the represented by the numeral 111 gets fixed over the surface 072-b. Since the surface 072-a is tapered, it allows the remaining part of the cannula to be retrieved easily. As the suture is attached internally to the tip, which is stuck in the cavity of the receiver unit, the suture essentially controls the endo-clamp via this receiver unit.

FIG. 32 depicts another embodiment of a locking element of a receiver unit, which works with the applicator shown in FIG. 20. After the surgeon positions the applicator near this element, the tip indicated by number 110 slide into the gap between the suture length 080 and the surface of the cannula 082, by way of rotating into it. Next the applicator is pulled back. The suture gets looped around the portion 114 after the applicator snaps past the locking mechanism 115.

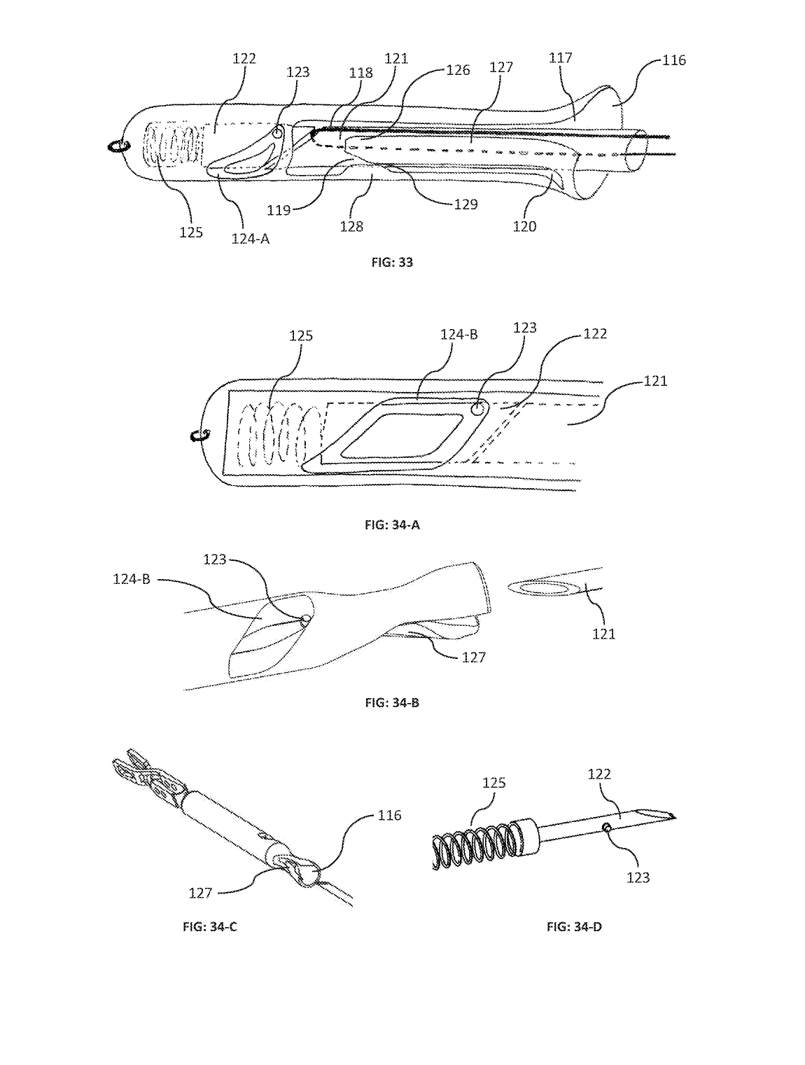

Illustrated in FIG. 33 is one embodiment of the receiver unit that typically is used with the applicator embodiment of FIG. 20. This unit comprises a cylindrical cavity with a smoothly opening flange generally represented by reference number 116. This allows the surgeon to easily slide in the applicator, which is represented by reference number 121, into the receiver unit. This unit has an opening represented by 117, through which the point 082 of the cannula 121 is aligned, such that the suture in the outer part of the cannula has sufficient space while the cannula slides through. As the surgeon pushes the cannula all the way inward, it pushes against a stopper, generally indicated by reference number 122. The stopper may be shaped so as to accept the cannula only in the correct orientation.

After this point, in some embodiments, the surgeon may manually rotate the cannula in order to transfer the suture onto the receiver unit, or they may utilize a spring-cam mechanism to effect this rotation automatically. In either case, a relative rotation between the cannula and the receiver unit effects a smooth transfer of the suture from the cannula to the receiver unit. How this transfer happens is explained subsequently. Also, in some embodiments where this is done automatically, the mechanism to do so is also explained subsequently.

FIG. 33 further illustrates a follower generally represented by reference number 123, which is attached to the stopper 122. A spring, generally indicated by reference number 125 is positioned behind the stopper 122. A characteristically shaped cam surface, which is generally represented by reference number 124-A, is carved out on the surface wall of the body of the receiver unit. Thus, when the cannula, represented by reference number 121, pushes further on the stopper 122, compressing the spring 125, the follower forces a relative rotational motion between the receiver unit and the cannula, about their coaxial axis.

FIG. 34-A illustrates an alternative shape of the cam surface 124-B that may also be used, depending upon the initial position of the stopper, the shape of the elements of the receiver unit, and other dimensions. However, the shapes shown in the figures generally illustrate the required shape of the cam, or the slots. Such a cam or slot mechanism may also be positioned on the other end of the cannula, instead of the receiver unit, thus rotating the cannula itself when it is pushed, rather than rotating the receiver unit. Either way, a cam surface or curved slots are utilized, to ultimately effect a relative rotation between the cannula and receiver unit. It may be noted that other mechanisms may also be employed to effect this same action of relative rotation.

FIG. 34-B, FIG. 34-C and FIG. 34-D represent alternative embodiments of the parts described in FIG. 33 and FIG. 34-A.

The structure of the receiver unit, as per FIG. 33 is explained as follows. It further includes a wing member generally represented by reference number 127. Wing 127 has a tip at the front, generally referenced by the number 126. This wing member leaves a gap 117 for the outer suture, represented by reference number 118 to pass. A small protrusion generally represented by reference number 128 rises around the curved surface below to nearly touch the wing 127, leaving a very small gap, generally indicated by reference number 129. The wing 127 and protrusion 128 curve towards the gap 129 in a gradual manner, giving rise to a V-shaped opening, generally represented by reference number 119. The wing 127 also leaves a slit 120 on the same side, in continuation to the opening 119 and thin gap 129.

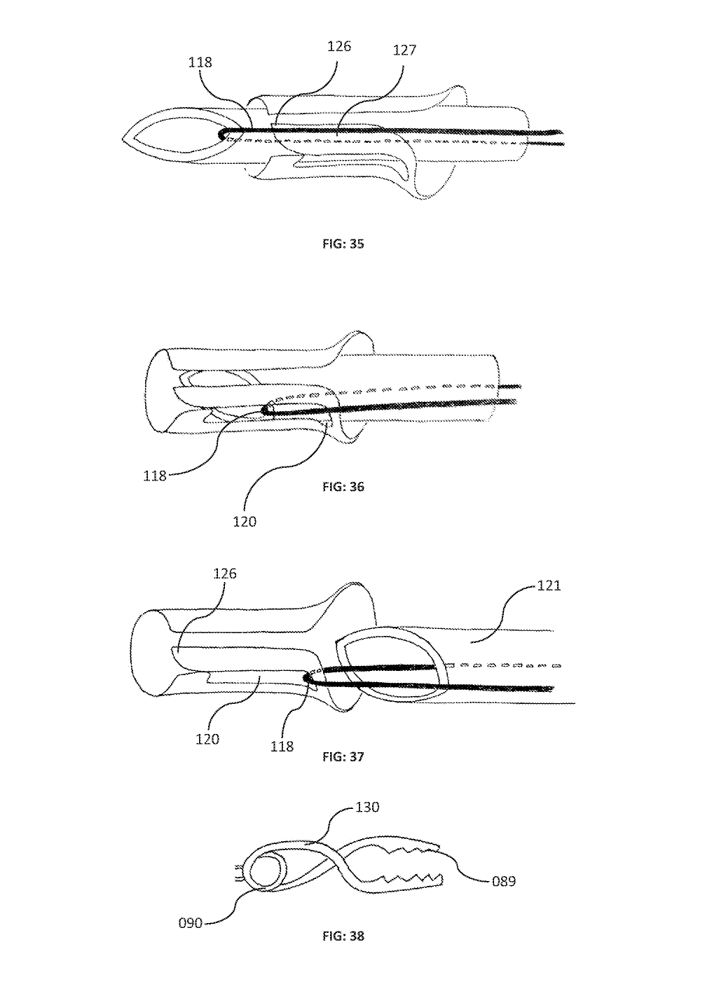

Referring to FIG. 35, a small amount of relative rotation between the cannula and receiver unit is illustrated. The point 082 of the cannula rotates beyond the tip 126 of the wing to position the suture 118 within the opening 119. This rotation also forces the outer segment of the suture 118 to climb over the wing 127. In some embodiments, the end 126 may have a pointed shape, the upper edge of wing 127 may be sharp and thin, and the inner surface of the wing 127 may be shaped to tightly fit the outer surface of the cannula, with a minimal gap. All these features enable the smooth sliding of the wing 127 under the outer part of the suture 118.

Once the point 082 is within the opening 119, the cannula along with the suture is pulled backward, either manually or due to the spring-cam mechanism. The outer suture 118 is forced through the thin gap 129 and into the slit 120. Once it is past the thin gap 129, the suture gets trapped, or looped within the slit 120, and thus onto the receiver mechanism. These two steps are shown in FIG. 36 and FIG. 37. The cannula can be pulled away, leaving the suture attached to the receiver unit. In some embodiments, the stopper 122 may be positioned such that it is in front of the thin gap 129, such that it provides for an additional locking mechanism, trapping the suture or control link loop within the slit 120.

Since the receiver unit is attached to the endo-clamp, which in turn has gripped the organ, pulling on the suture outside the body results in retraction of the organ. As described earlier, the suture can now be fastened at a desired length outside the skin surface by a clip and the surgery may proceed. If the surgeon so desires to retract the organ in a different direction, another cannula with suture may be introduced from the new desired direction, and attached to the same receiver unit, along with the earlier suture. Now, it is possible to manipulate the endo-clamp in either direction to achieve the new desired direction and extent of retraction.

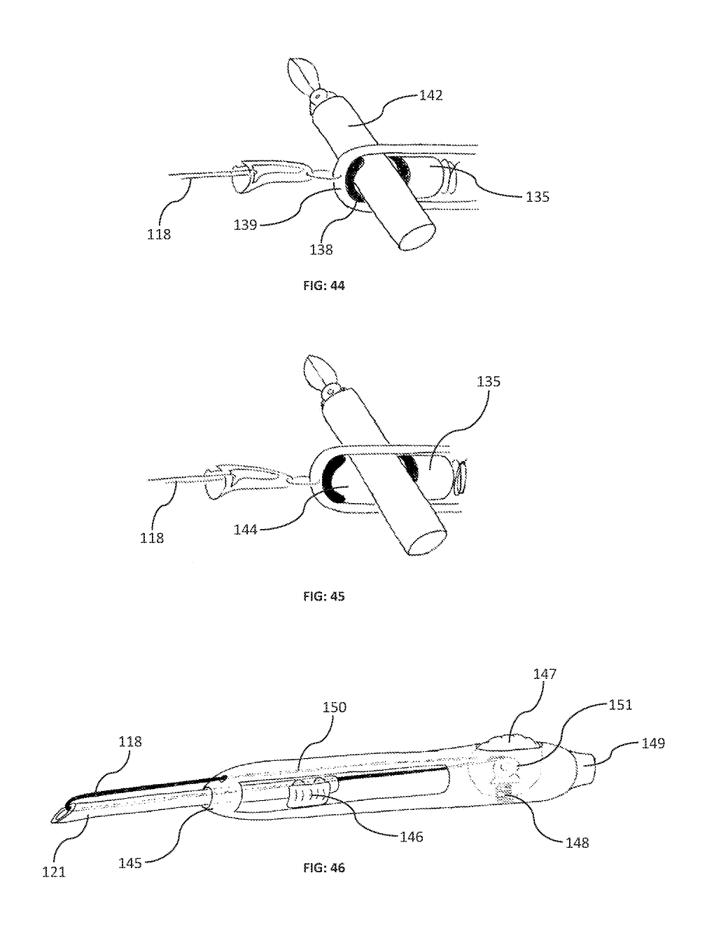

On the outer side of the body, the surgeon may use simple clips to hold the sutures and hence the organs retracted. Similarly, the cannula, once the attachment work is done, may be discarded. However, to make things more user-friendly, an applicator housing may be used to manipulate these components. In some embodiments, such an applicator may be pen-like in shape so as to make it easy to hold and use. Such a device is illustrated in FIG. 46. It includes the cannula 121 protruding outwards, in a manner that is similar to the lead of a clutch-pencil or a mechanical pencil. Attached to the cannula is a thumb handle, generally represented by reference number 146, which can be used to extend or retract the cannula between inside and outside the pen-like housing. This thumb handle may include a push-locking mechanism, such that the position of the cannula may be fixed. It may be released on pressing the handle, and thus moving it. This mechanism may be similar to that used on a cutting blade. Alternatively, the said thumb handle may be motorized by means of a linear motor, or any type of a linear actuation mechanism, such that this motion may be electronically controlled.

Generally represented by reference number 145 is the tip of the pen-like applicator housing, which smoothly narrows down from where the cannula and the suture 118 project out. Both sides of the suture 118 inside and outside the cannula continue inward along the length of the pen-like applicator housing, generally represented by reference number 150. They eventually wind around a pulley, or bobbin at the base of the pen-like applicator housing, which is generally represented by reference number 151. This bobbin is connected to an ergonomically positioned wheel 147. In some embodiments, this wheel may be made of a material having a good grip. A spring at the opposite end, generally represented by reference number 148, keeps the wheel pushed against the opening in the pen-like applicator housing body, thus keeping it locked and preventing it from moving. Thus, the sutures are prevented from getting extended. Whenever a user wishes to retract or loosen the suture length, the wheel is pressed downward, to release this ‘lock’, after which it can be freely rotated. In other embodiments, a different type of locking and retracting or releasing mechanism may be used for handling the sutures. In yet other embodiments, this wheel or bobbin may be motorized such that the control link can be wound up or released using electronic controls and a power source such as electrical power or pneumatic power.

The pen-like applicator housing may further include a push button at the back, generally represented by reference number 149. This push button may be connected to a cam-slot-spring mechanism similar to the one explained in FIG. 33 and FIG. 34-A. Thus, in the end position of the cannula, this button can be pushed in order to effect the relative rotation between the cannula and the receiver unit.

Referring to FIG. 38, we see one embodiment of the endo-clamp, which is similar to a bulldog clamp, with a serrated jaw generally represented by reference number 089, a spring at its base generally represented by reference number 090 and a gripping position generally represented by reference number 130. The position 130 may be used by specialized graspers to open the jaws of the clamp. The spring keeps the jaws of the clamp closed.

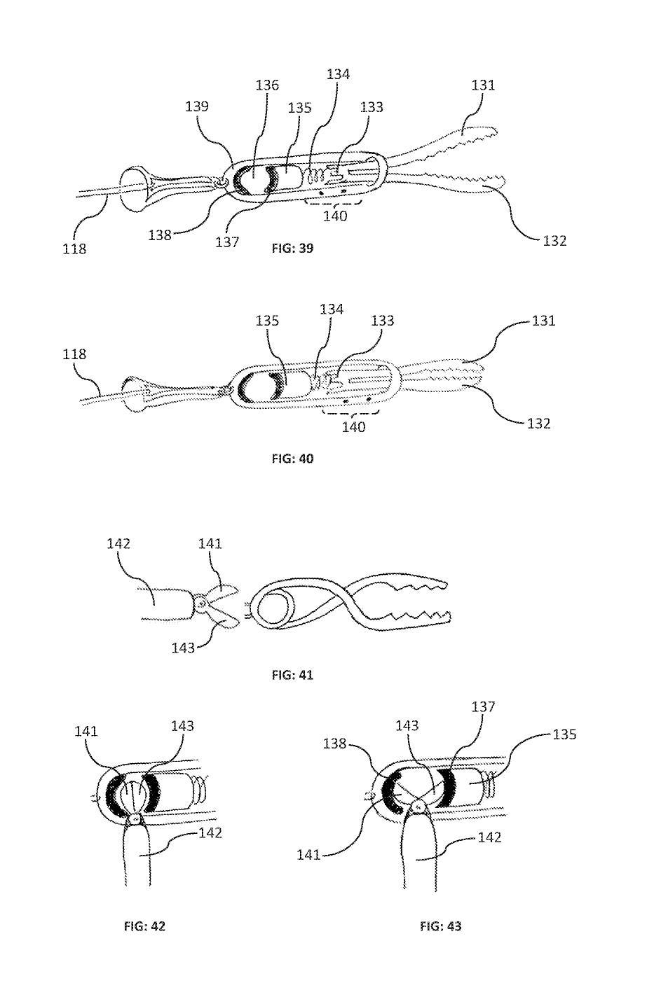

FIG. 39 and FIG. 40 show another embodiment of the endo-clamp with the jaws in open and closed positions respectively. In this embodiment, a different mechanism is used to open and close the jaw. It includes a frame generally represented by reference number 139. Internally, it includes a push-button, generally represented by reference number 135. On the inner surfaces of the push button and the frame are rubber-like linings for better grip, which are generally represented by reference numbers 137 and 138 respectively. These may be made of silicone, biocompatible polymers, or other suitable material that can provide a good grip. In this embodiment, the jaws, which are generally represented by reference numbers 131 and 132 are connected together at a common base, generally represented by reference numeral 133. This base further includes cams, slots and connects to the push button via a spring which is generally represented by reference number 134. Collectively, these components are referred to by the number 140 and they perform the function same as that found in ordinary ‘click ball-pens’ such that each ‘click’ or push of the button 135 causes the jaws 133 to switch between a forward position and a retracted position. In the forward position, the jaws remain open, and in the retracted position, the jaws remain closed, as illustrated in FIG. 39 and FIG. 40 respectively.

The need for using this concept uniquely in this situation is part of the inventive step of this system. Other systems that use an internal endo-clamp for organ retraction, must rely on specialized graspers in order to open or close the jaws. This is illustrated in FIG. 41, where a grasper, indicated by reference number 142 attempts to open an endo-clamp with its jaws, generally represented by reference numbers 141 and 143. If, during the middle of a procedure, there is felt a need to re-position the endo-clamp, to grip another organ, or another part of the same organ, the procedure has to be stopped. One of the instruments removed, and the special grasper used for manipulating the endo-clamp inserted and then the endo-clamp can be re-positioned. Thus, the ongoing procedure gets interrupted in order to re-position the tissue.

Further, in systems where the endo-clamp is hooked internally on the inner surface of the abdominal wall, one of the surgical instruments must be used in order to reposition the location of this hook.

By implementing a push-button ‘click ball-pen’ like mechanism to actuate the grasper, we eliminate the need for a specialized grasper to handle the endo-clamp. As illustrated in FIG. 42 and FIG. 43, any instrument that has a pair of large and strong enough jaws can be used to actuate the push button, and hence may be used to manipulate the endo-clamp. The instrument may not even be a grasper, let alone specialized grasper. Even a scissor, with strong enough jaws can be used to push against the push button to actuate the endo-clamp.

If the only instruments available are without openable jaws, such as a hook or a monopolar cautery, or a suction-irrigation device, a second method may be used. Alternatively, it may also be used even if an instrument with jaws is available, but the jaws are too small or too weak to push against the button. This second method is illustrated in FIG. 44 and FIG. 45. Any available instrument is inserted into the gap 144. A suture is attached to the receiver unit by one of the methods mentioned earlier. When both ends of the suture 118 are pulled outwards, they result in the instrument pushing against the button 135, thus resulting in a toggle action on part of the endo-clamp.

FIG. 47 and FIG. 48 refer to another embodiment of the endo-clamp implementation where specialized instruments will not be necessary to actuate the jaws of the endo-clamp. This embodiment consists of a frame similar to a cylindrical pipe, with a saw-tooth surface lining it internally, said saw-tooth surfaces being generally indicated by reference number 152. The jaws of this endo-clamp are made of compliant plastic material which has spring-like properties. The two jaws are fixed to the frame by a hinge, generally represented by reference number 155. The other end of the jaw components is connected to a receiver unit, generally represented by reference number 003. Along the length of the jaw arms is also a protrusion, generally indicated by reference number 153. These components together constitute the ratchet-like mechanism to achieve opening and closing of the jaws and their locking in either position.

Initially, the jaw is kept in the open position due to the spring action of the compliant plastic material of the jaws. This force is generated by the bent section generally represented by reference number 154. This position is illustrated in FIG. 47. When the receiver unit pulls the grasper jaws away from the hinge 155, the jaws begin to close, while at the same time, the protrusion 153 is pulled and gets locked in successively further steps of the saw-tooth surface 152. This position, where the jaws are partially closed, is illustrated in FIG. 48. Thus, the further the receiver unit is pulled, the tighter the grip gets on the jaws.

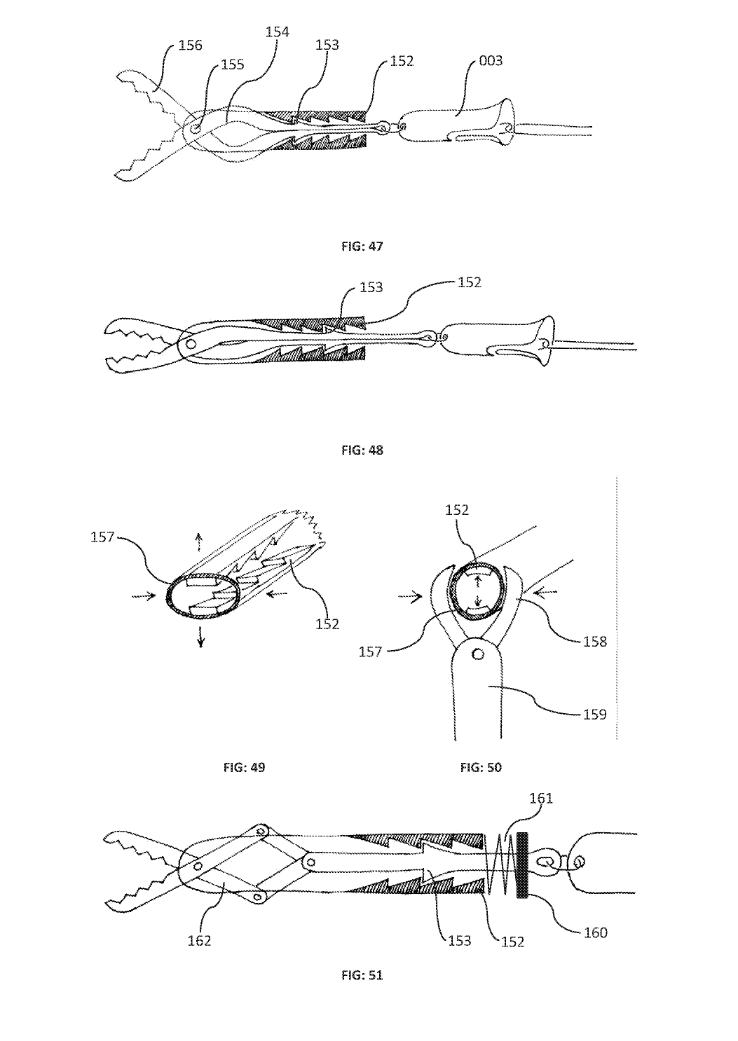

One method of releasing the jaw grip is illustrated with the help of FIG. 49 and FIG. 50. As shown, the cross section of the frame of the endo-clamp is oval in shape, such that the saw-tooth surfaces are on either side of the longer diameter. This cross section is generally represented by reference number 157. The material of the frame is also semi-rigid, such that it is strong, yet flexible to change the shape slightly.

A regular grasper, or a similar such device is generally illustrated by reference number 159 in FIG. 50. The jaw of such an instrument is also shown, generally represented by reference number 158. As shown in the figure, when the jaws of instrument 159 compress the cross section 157 along its longer diameter, the shape of the frame is altered, thus increasing the gap between the two saw-tooth surfaces. This frees up the protrusion 153 that had been locked at one of the steps of the saw-tooth surface 152. Once this locking mechanism is free, the spring action of the compliant material bent section 154 forces the jaws to open again. We can see that other similar mechanisms may also be employed which can lock and unlock the jaws of such a grasping device open and close.

Shown in FIG. 51 is another embodiment of a similar principle as described in FIG. 47 and FIG. 48. However, in this case, the jaws are made by a rigid material which is generally represented by reference numeral 162. In this embodiment, there is also a spring, generally represented by reference number 161 and a spring support structure generally represented by reference number 160. The spring and the spring support keep the jaws of this grasper open, unless they are locked by the ratchet-like mechanism described above. Such a saw-tooth surface is also shown in FIG. 51, indicated by reference number 152, on which a protrusion 153 gets locked. The cross section of this mechanism too, is similar to the one shown in FIGS. 49 and 50. The method to unlock is also the same, likewise.

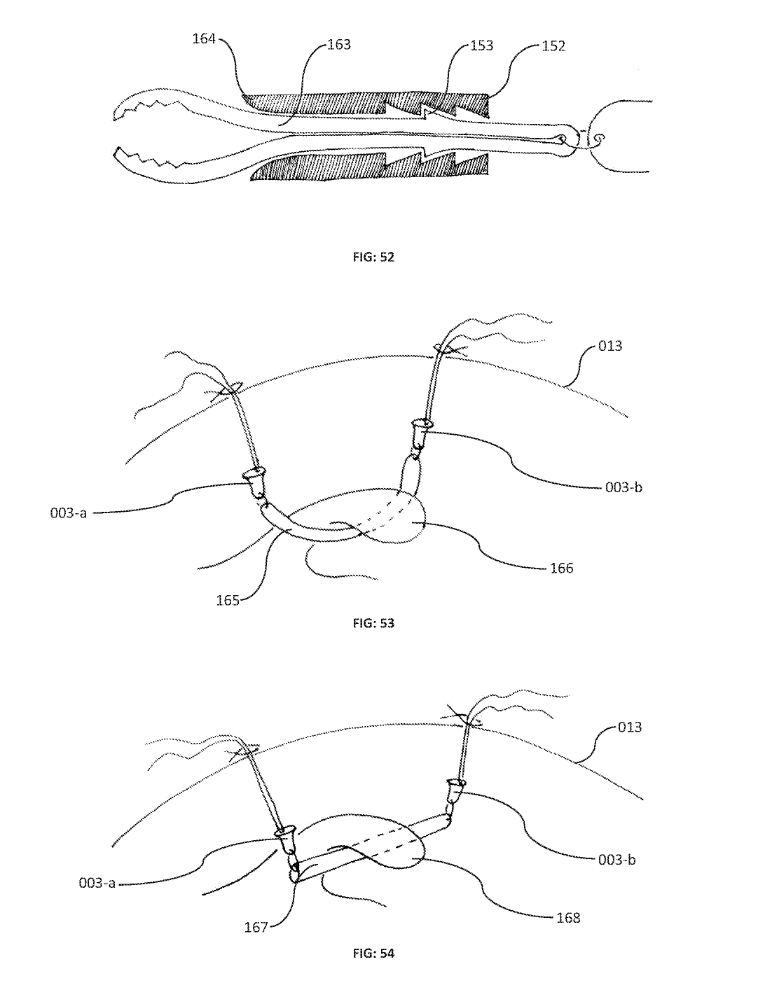

FIG. 52 illustrates yet another mechanism of implementing the endo-clamp where a similar saw-tooth surface 152 locks the protrusion 153 in various positions. In this case as well, the jaws, generally indicated by reference number 163 are made of a flexible but strong compliant material, which causes the jaws to tend to open up. However, when they are pulled inward by the receiver unit attached, they get closed due to the splayed mouth of the frame, generally represented by reference number 164.

Shown in FIG. 53 is a method of retracting larger organs by use of a flexible silicone band or a similar component, which is generally represented by reference number 165. The organ being retracted is generally represented by reference number 166. Typically, one or more receiver units may be attached to various parts of the flexible band, such as its two ends. These are generally illustrated by reference numbers 003-a and 003-b in FIG. 53. Sutures connect to these receiver units, which may be controlled from outside the body since they pass through the tissue surface 013.

As an alternative to the soft silicone band, a rigid rod may also be used for similar purposes, as indicated by reference number 167 in FIG. 54. In some embodiments, this may be a telescoping rod capable of retracting a wide range of sizes of organs. This may be especially useful for a large and heavy organ, generally represented by reference number 168.

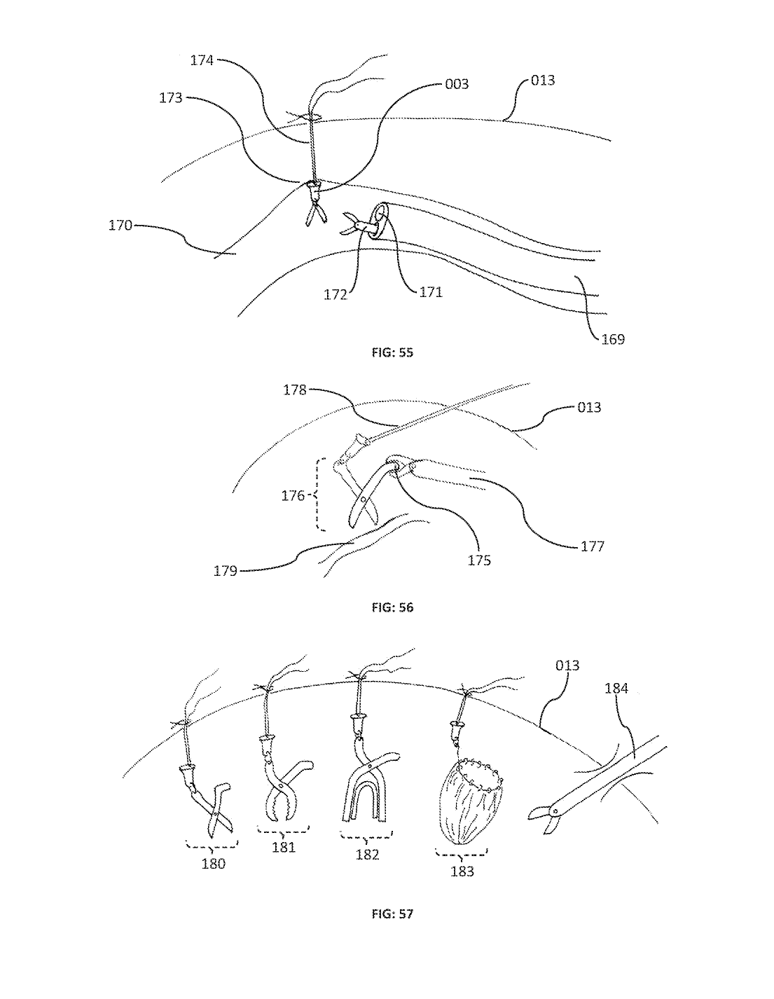

As yet another use of this device, it may be inserted in various body cavities, tubes or tracts, especially in which surgical procedures are carried out. These may include the tracheal tube, the gastro-intestinal tract, or even larger blood vessels which require some sort of interventions. One such arrangement is illustrated in FIG. 55, where the tract is generally represented by reference number 170. A set of surgical instruments may be inserted in such a tract for carrying out a surgical procedure. In this illustration, a single-channel endoscope is shown as an example, which is generally illustrated by reference number 169. It has a camera and one instrument in the channel, generally represented by reference numbers 171 and 172 respectively. This instrument may be a grasper, which helps carry the receiver unit in the tract.

Once at the surgical site, the receiver unit may get connected to an external suture, as represented by reference number 174. By applying force on the suture externally, a physician may indirectly apply force on the tract boundary, and in some cases, alter the position of the tract 170 itself.

Alternatively, another instrument may be added to the receiver unit, such as a scissor, or a clip applier, which can be actuated in combination with the existing grasper and the external force applied by the suture. This concept is further explained with help of FIGS. 56 and 57.

As shown in FIG. 56, the receiver unit is connected to a specially designed instrument tip, such as a scissor. One jaw of the instrument directly connects to the receiver unit, while the other jaw has a protrusion at the base, generally represented by reference number 175. This protrusion can be used to control and position the instrument tip, by using an existing grasper, generally illustrated by reference number 177. This entire assembly is generally represented by reference number 176. A tissue which may be manipulated by this method is also shown below, generally represented by reference number 179.

The overall benefit in using this method is that additional instruments may be introduced and used within the body cavity without requiring more number of large incisions or ports. These additional instruments can be controlled by a combination of a grasper which is already inside, and a suture that can pass through the skin. This may be especially useful for instruments which are important, but used less frequently than usual instruments like regular graspers and scissors. One example is that of clip appliers in surgeries which require only one or two or few clips to be applied. Another example is an organ retrieval bag which may be used only occasionally during the surgery.

Some such examples are illustrated in FIG. 57, which gives a picture of how a large variety of instruments may be used inside the body cavity, while still making a fewer number of ports and smaller size of incisions. These examples include the scissor, grasper, clip applier and tissue retrieval bag, which are generally represented by reference numbers 180, 181, 182 and 183 respectively. Each of these instruments may be actuated in combination with an existing grasper and the force externally applied using the control link, which may be a suture. For example, the scissor or grasper jaws may be opened or closed using this method. In case of the clip applier, the action of actual application of the clip may be carried out by this method. In case of an organ retrieval bag, it may be opened or closed from outside the body by pulling on the suture that passes through the skin. Thus some of the more complex procedures may also be carried out by reduced port laparoscopy methods, or by single port laparoscopic surgery using these more sophisticated instruments.

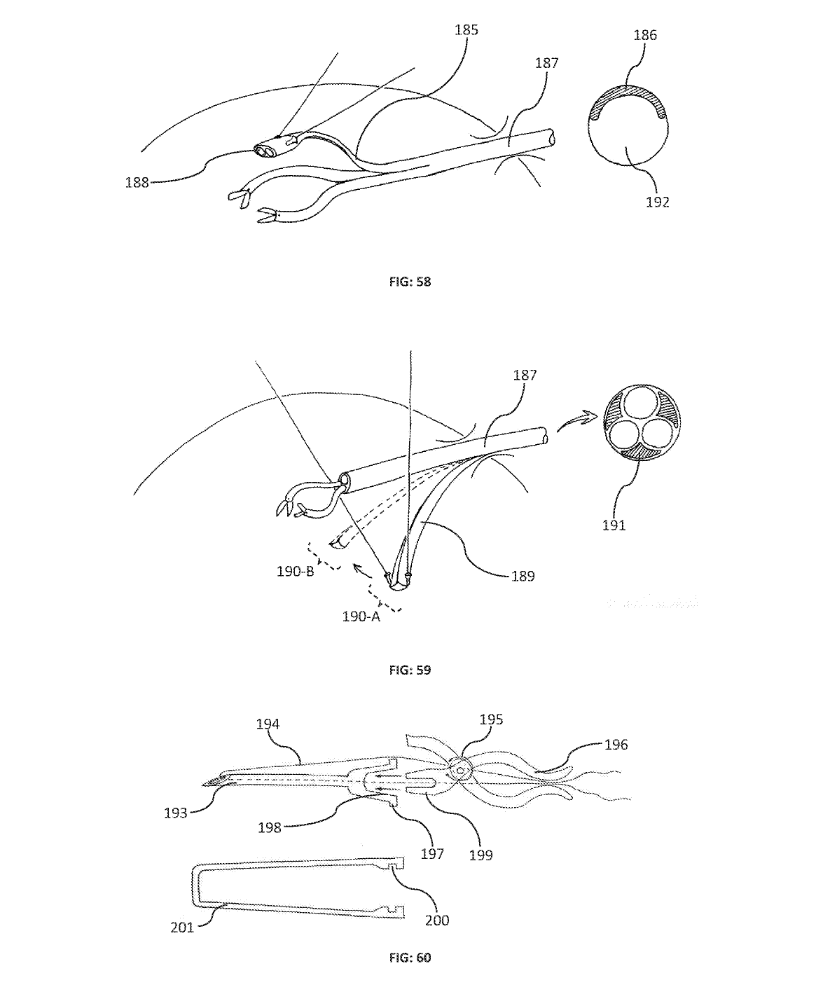

Taking this concept even further, shown in FIG. 58 is an illustration of how the receiver units may be attached to a camera of a reduced or single port surgery mechanism. The camera, generally indicated by reference number 188, is hoisted on a thin U-shaped semi-flexible flap, which is generally indicated by reference number 185. The cross section of this flap is generally indicated by reference number 186. This leaves a large cross-sectional area free to accommodate other instruments and mechanisms. This additional area is generally indicated by reference number 192. Further, the curved shape of the flap tends to keep the camera pushed downward, while the sutures attached to it through the skin can pull it upward to a desired height. Multiple sutures may be used to orient the camera in the correct direction. The advantage of this method is that it requires only a thin U-shaped attachment of the camera to the main surgical shaft, thus minimizing the cross-sectional diameter of the device. This would reduce the size of the port, leading fewer complications, such as port-site hernia, pain, and so on. Alternatively, it would leave more cross sectional area to accommodate other instruments, enabling more sophisticated surgeries to be performed.

Illustrated in FIG. 59 is yet another application of this apparatus and the receiver unit, for fluid flow instruments like suction or irrigation. Such an instrument for fluid flow is generally illustrated by reference number 189. This instrument is attached to a bundle of instruments for single port or reduced port surgery. This bundle, which may be a robotic arm, or a manual use assembly, is generally represented by reference number 187. The instrument 189 may be spring-loaded such that it tends to move downwards to a lower position, generally indicated by reference number 190-A, which is into the surgical site for delivering fluid or suctioning fluid, depending upon the operation. It may also be attached to one or more receiver units and hence one or more external sutures which can apply a force on the instrument in various directions. These sutures may thus pull the instrument 189 to a second position, generally represented by reference number 190-B.

The cross-section of the bundle of instruments is generally represented by reference number 191, pointing out the areas which are odd-shaped and cannot be used for any standard instruments. However, an advantage of using the receiver units and externally controlled sutures onto such a spring-loaded instrument 189 is that within the available space, additional functions of suction and irrigation may be accommodated.

In FIG. 60 are illustrated various accessory components that enable the convenient use of this apparatus by just one hand, while at the same time ensuring safety of the user as well as the patient. The applicator, which in this embodiment is shown as a cannula is generally represented by reference number 193. The control link, which may be a thin filament-like structure such as a suture, is represented by reference number 194. The cannula has a pointed tip on its distal end around which the suture is positioned, as shown in FIG. 60. On the proximal end of the cannula is an opening with a female luer taper on its inner surface, which is generally represented by reference number 198. On the outer surface are protrusions which form a male-thread or a tabbed hub, and are generally represented by reference number 197.

Also illustrated in FIG. 60 is an outer clipping component with a spring, which is generally represented by reference number 195. The outer clip also has ergonomically shaped jaws which are generally represented by reference number 196. The spring keeps the jaws in the closed position. The jaws may be such that they are easy to grip and hold by a human finger from the outside, while have serrations or gripping surfaces to hold on to the control link, such as a suture, on the inner side. Generally indicated by reference number 199 is a male luer taper at the base of the outer clip, on one of its jaws.

Also illustrated in FIG. 60 and generally represented by reference number 201 is a hollow tube which is used as a safety holder for the applicator such as a needle or cannula, such that it can be stored safely after use. At the opening of this safety holder is sleeve with a female thread, generally represented by reference number 200.

As shown in the FIG. 60, the outer clip is attached to the applicator by means of the male and female luer tapers. The suture is routed along the inner and outer lengths of the applicator, around its distal end, and held firmly by the spring loaded jaws of the outer clip on the proximal end. The surgeon introduces the applicator into the patient's body by holding the outer clip at the ergonomically designed jaws 196. When the suture has been transferred onto the receiver unit inside, the surgeon gently opens up the jaws of the outer clip by pressing on the portion of the jaws on the opposite side of the spring, to release the sutures from the grip of the outer clip. While doing so, the applicator is withdrawn from the patient's body completely, leaving only the pair of suture ends projecting out of the surface.

The surgeon may quickly insert the applicator inside the safety hold 201, and rotate it such that the male threads 197 latch onto the female threads 200. At this point, the surgeon may pull the outer clip free off the applicator, as the luer taper grip between the outer clip and applicator is weaker than the threaded grip between the safety holder and the applicator. Thus, the applicator is stored safely into the safety hold 201, helping avoid needle prick injuries for the users. Further, the same outer clip may now be used to hold the sutures in position against the outer surface of the skin, in order to hold them in place.

These components thus enable the use of the entire apparatus in a convenient manner, using only 2 fingers of one hand of a user. At the same time, it also ensures the timely removal and storage of the applicator in a safe and easy manner so as to avoid injury to the patient or the surgeon.

We thus see that many combinations of the above factors give rise to different bestcases of devices. However, the general principle of the utilization thin sutures or filaments to control internally placed surgical manipulators, such as grasping devices, remains the same. The ability to on-the-go elect to attach one or more such sutures from outside the body to the endo-clamp or other instruments inside the body in a convenient manner, forms the core inventive step of this invention. Many other embodiments apart from the ones described above may also result in similar such results, and ultimately allow the surgeon to perform increasingly complex procedures with fewer and smaller incisions.

As will be realized, the present invention is capable of various other embodiments and that its several components and related details are capable of various iterations, all without departing from the basic concept of the present invention. This may include various materials, alternate mechanisms, etc. Accordingly, descriptions will be regarded as illustrative in nature and not as restrictive in any form whatsoever. Modifications and variations of the system and apparatus described herein will be obvious to those skilled in the art. Such modifications and variations are intended to come within the scope of this patent.

1. A surgical apparatus comprising:
a. a surgical end effector component on a first side of a tissue surface,
b. an external control link on a second side of said tissue surface, said link having a proximal end and a distal end,
c. an applicator carrying said external control link capable of conveying the distal end of the link across said tissue surface from the second side to the first side,
d. at least one receiver unit attached to said surgical end effector, said receiver unit comprising means for:
i. transferring said distal end of said control link from said applicator, to said receiver unit, and
ii. mechanically locking said distal end of said control link to said receiver unit,
whereby, said external control link may manipulate said receiver unit and hence said surgical end effector by exerting forces on them from said second side of said tissue surface in order to perform surgical tasks on said first side of said tissue surface.

2. A surgical apparatus according to claim 1 wherein, said surgical end effector is a mechanical grasping device whereby said grasping device may grasp and retract various organs in a patient's body.

3. A surgical apparatus according to claim 2 wherein,
a. said apparatus further includes a second instrument for manipulating the grasping device,
b. said mechanical grasping device further includes means for actuating the device,
whereby, the grasping device may be operated by exerting a force on the external control link, while being held in place by said second instrument.

4. A surgical apparatus according to claim 1 wherein, said surgical end effector is a flexible band, whereby the band may loop around organs.

5. A surgical apparatus according to claim 4 wherein, the assembly further includes a plurality of said receiver units attached to said flexible band at multiple locations, whereby the band may exert forces on organs from multiple directions.

6. A surgical apparatus according to claim 1 wherein, said surgical end effector is a telescoping rod, further including a plurality of said receiver units whereby said rod may manipulate large organs in a patient's body in multiple directions.

7. A surgical apparatus according to claim 1, wherein, said external control link is a strong, thin, filament-like flexible structure such as a suture, wire-rope or thread.

8. A surgical apparatus according to claim 7 wherein, the filament structure is thin enough in diameter such that it causes no visible scar on said tissue surface.

9. A surgical apparatus according to claim 1 wherein said applicator is a rigid hollow cannula.

10. A surgical apparatus according to claim 9 wherein said cannula is thin enough in diameter such that it causes no visible scar on said tissue surface.

11. A surgical apparatus according to claim 9 wherein, said control link is held by said cannula such that the control link is bent in two sections at the distal end of said cannula, a first section passing through the hollow inside of said cannula and a second section passing along the outer wall of said cannula.

12. A surgical apparatus according to claim 11 wherein, said receiver unit comprises:
a. a cylindrical cavity in which said cannula may smoothly slide and rotate in,
b. a wing carved out on the side wall of said receiver unit forming a slit,
whereby, said control link is smoothly guided over said wing and into said slit of said receiver unit, getting mechanically locked around said slit.

13. A surgical apparatus according to claim 12 wherein said slit of said receiver unit is large enough to allow locking of multiple control links onto a single receiver unit, whereby multiple control links are able to apply force on said control link from multiple directions.

14. A surgical apparatus according to claim 12 wherein said receiver unit further comprises:
a. a stopper capable of translating and rotating within said receiver unit for guiding the cannula,
b. a spring to keep said stopper pushed forward,
c. a cam and follower mechanism for rotating said stopper,
whereby, said stopper forces said cannula to slide and rotate in the cylindrical cavity, as said cannula is pushed against said stopper, whereby, said control link is smoothly guided over said wing, locking the link into said slit.

15. A surgical apparatus according to claim 9 wherein, said apparatus further includes a safety holder for said cannula, whereby cannula tip is stored safely after use.

16. A surgical apparatus according to claim 1 wherein the apparatus further includes an outer clip for fastening said control link on said second side of said tissue surface, whereby the link is held firmly in place, keeping a constant tension force applied through said control link.

17. A surgical apparatus according to claim 16, wherein
a. said apparatus further includes a safety holder, and
b. said outer clip is attached to said applicator by friction of a pair of luer taper surfaces, and
c. said applicator gets attached to said safety holder by thread locking,
whereby the entire procedure can be carried out by a human user with only one hand by transferring said applicator from being held by said outer clip, to being secured in said safety holder.

18. A surgical apparatus according to claim 1 wherein said surgical end effector is a shearing instrument whereby, said external control link causes actuation of said shearing component.

19. A surgical apparatus according to claim 1 wherein said tissue surface is an abdominal wall, whereby the apparatus may be useful for laparoscopic surgery.

20. A surgical apparatus according to claim 1 wherein said apparatus further includes,
a. an applicator housing,
b. means for driving said applicator forward and backward,
c. means for winding and unwinding said control link,
whereby, said applicator housing may be used for deploying said applicator and control link and may be controlled electronically along with a surgical robot.

21. A method for manipulating surgical end effectors comprising the steps of:
a. inserting said surgical end effector on a first side of a tissue surface,
b. introducing a control link having a distal end on said first side of tissue surface while a proximal end remains on a second side of said tissue surface,
c. connecting the distal end of said control link to said end effector,
whereby, said end effector is manipulated from across said tissue surface, by exerting a force on the proximal end of said control link.

22. A method according to claim 21, wherein the surgical end effector is an atraumatic tissue grasping component.

23. A method according to claim 22, wherein said control link is a thin suture such that it does not cause any visible scar on the tissue surface through which the suture passes.
